(19) United States
(12) Patent Application Publication
(10) Pub. No.: US 2009/0011474 A1
(43) Pub. Date: Jan. 8, 2009

(54) Process for producing sugars from cellulosic biomass

(75) Inventors: Venkatesh Balan, East Lansing, MI (US); Bruce E. Dale, Mason, MI (US)

Correspondence Address: IAN C. McLEOD, P.C., 2190 Commons Parkway, Okemos, MI, 48864, US

(73) Assignee: Board of Trustees of Michigan State University, East Lansing, MI (US)

(21) Appl. No.: 12/214,687

(22) Filed: Jun. 20, 2008

Related U.S. Application Data
(60) Provisional application No. 60/936,509, filed on Jun. 20, 2007.

(51) Int. Cl. C12P 7/06; C12P 19/14; C12P 7/04

(52) U.S. Cl. 435/99; 435/157; 435/161

(57) ABSTRACT

A process for increasing production of sugars from cellulose in a plant biomass using ammonia after swelling of the biomass with water and enzymatic hydrolysis is described. The sugars are preferably fermented to an alcohol, particularly ethanol as a fuel for vehicles.

### CROSS-REFERENCE TO RELATED APPLICATIONS

This application claims benefit to U.S. Provisional Application Ser. No. 60/936,509, filed Jun. 20, 2007, which is incorporated herein by reference in its entirety.

### STATEMENT REGARDING FEDERALLY SPONSORED RESEARCH OR DEVELOPMENT

Not Applicable.

### REFERENCE TO A “COMPUTER LISTING APPENDIX SUBMITTED ON A COMPACT DISC”

Not Applicable.

### BACKGROUND OF THE INVENTION

(1) Field of the Invention

The present invention relates to an improved process, wherein a plant biomass is pre-soaked in water for a period of time so as to enhance sugar production using concentrated ammonia to disrupt the biomass and then enzymatic hydrolysis of the biomass. In particular, the present invention relates to soaking plant biomass in water and then ammonia treatment in order to improve yields of sugars from carbohydrates in the biomass using enzymatic hydrolysis, wherein the enzymes hydrolyze cellulose, hemicellulose and other carbohydrates.

(2) Description of the Related Art

The growing U.S. appetite for petroleum, together with demand growth in China, India, and the rest of the world, has pushed prices to new highs. The United States uses over 20 million barrels of petroleum per day, of which 58% is imported. Prices of oil are significant and continue to rise. Bioethanol is one of the low cost, consumer-friendly ways to reduce gasoline consumption and carbon dioxide emissions from vehicles. It is a clean fuel that can be used in today's cars. One of the many attributes of bioethanol is that it does not contribute net carbon dioxide to the atmosphere. (See e.g., Ohara, H., Biorefineryi, Appl Microbiol Biotechnol, 2003, 62, 474-477.) Thus, a considerable amount of research is underway to produce ethanol from the cellulose and hemicellulose in plants, which are far more abundant in nature and cheaper to produce. (See e.g., Ragauskas, A. J., Williams, C, K., Davison, B. H., Britovsek, G., Cairney, J., Eckert, C. A., Frederick Jr., W. J., Hallett, J. P., Leak, D. J., Liotta, C. L., Mielenz, J. R., Murphy, R., Templer, R., and Tschaplinski, T., The Path Forward for Biofuels and Biomaterials, Science, 2006, 311, 484-489); and (Gray, K. A., Zhao, L., and Emptage, M., Bioethanol, Current Opinion in Chemical Biology, 2006, 10, 141-146.)

Enzymatic hydrolysis of cellulosic biomass generally results in low sugar and resulting yields unless the biomass undergoes a pretreatment step. A pretreatment method to improve the efficiency of the hydrolysis is an ammonia fiber explosion (AFEX) process as described in U.S. Pat. Nos. 4,600,590 and 6,106,888.

U.S. Pat. No. 4,600,590 to Dale, the disclosure of which is herein incorporated by reference for all purposes, discloses a method of treating cellulose-containing materials to increase chemical and biological reactivity of cellulose. The cellulose is contacted, in a pressure vessel, with a volatile liquid swelling agent having a vapor pressure greater than atmospheric at ambient temperatures, such as ammonia. The contact is maintained for a sufficient time to enable said agent to swell the cellulose fibers. The pressure is rapidly reduced to atmospheric, allowing said agent to boil and explode the cellulose fiber structure. The rapid pressure reduction also causes some freezing of the cellulose. The agent is separated from said cellulose and recovered for recycling.

U.S. Pat. No. 6,106,888 to Dale et al., the disclosure of which is herein incorporated by reference for all purposes, discloses a process for treating cellulosic material. The process includes a screw in barrel apparatus for expanding cellulosic materials. The expanded cellulosic material is useful as an animal feed and a nutrient source for fermentation processes.

Typically, AFEX includes adding liquid ammonia to a biomass under high pressure (200-500 psi) and moderate temperatures (60-200° C.) before rapidly releasing the pressure. This process compares favorably economically to other leading pretreatment methods, and continued research has further improved its economics. (See e.g., Eggeman, T., and Elander, R. T., Process and economic analysis of pretreatment technologies, Bioresource Technol., 2005, 96, 2019-2025.) AFEX decrystallizes cellulose, hydrolyzes hemicellulose, removes and depolymerizes lignin and exposes additional fiber surface area.

Cellulose, one of the major component in plant cell wall is a linear condensation polymer consisting of D-anhydroglucopyranose joined together by β-1,4-linkage with a degree of polymerization from 100 to 20,000. Adjacent cellulose molecules are coupled by extensive hydrogen bonds and van der Waals forces results in a parallel alignment and a crystalline structure, which produces a straight, stable heterogeneous supramolecular structure and low accessibility to chemicals and cellulases. (See e.g., Mantanis, G. I., Young, R. A., and Rowell, R. M., Swelling of compressed cellulose fiber webs in organic liquids, Cellulose, 1995, 2, 1-22.) Disruption of these bonds and forces by swelling, will generate internal stress and improve the accessibility to chemicals like ammonia during pretreatment process. (See e.g., Ye, D., and Farriol, X., Improving accessibility and reactivity of celluloses of annual plants for the synthesis of methylcellulose, Cellulose, 2005, 12, 507-515; and Boluk, Y. Acid-base interactions and swelling of cellulose fibers in organic liquids. Cellulose 2005, 12, 577-593.)

With a strong cellulose swelling agents, it is possible to reach a critical point where the entire crystalline structure of the fiber is disrupted and the fiber structure is lost. (Zhang, Y.-H. P., Cui, J., Lynd, L R., and Kuang, L. R., A Transition from Cellulose Swelling to Cellulose Dissolution by o-Phosphoric Acid: Evidence from Enzymatic Hydrolysis and Supramolecular Structure, Biomacromolecules, 2006, 7, 644-648.) Three important parameters were found to be important to cellulose swelling: (1) the hydrogen bonding fraction, (2) solvent molar volume, and (3) the cellulose structure. The width and distribution of voids and the lateral-order spectrum were especially important (See e.g., Mantanis, G. I., Young, R. A., and Rowell, R. M., Swelling of compressed cellulose fiber webs in organic liquids, Cellulose, 1995, 2, 1-22). The swelling will be more predominant when there is electrostatic repulsion of the ionic surfactants adsorbed on to the cellulose polymer chains. (Bahar, H., Okubayashi, S., and Bechtold, T., Splitting tendency of cellulosic fibers, Part 2: Effects of fiber swelling in alkali solution, Cellulose, 2006, 13, 403-409; and Uraki, Y., Takeshi Imura, T., Kishimoto, T., and Ubukata, M., Boday temperature-responsive gels derived from hydroxypropylcellose bearing lignin II: adsorption and release behavior, Cellulose, 2006, 13, 225-234.)

Miscanthus (Miscanthus×giganteus), also known as Giant Chinese Silver Grass, is a large perennial rhizomatous grass utilizing the C4 biosynthesis pathway (FIG. 7A). It can grow over 12 feet tall and has attracted considerable attention in Europe and the US as a possible dedicated energy crop, either as fuel for electricity generation or, more recently, for conversion to a biofuel such as ethanol. Miscanthus has numerous favorable characteristics as a dedicated energy crop. It can be grown in poor quality soil with a high yield, and requires little herbicide, nitrogen, and water. (See e.g., Clifton-Brown, J. C.; Lewandowski, I.; Andersson, B. Performance of 15 Miscanthus genotypes at five sites in Europe. Agronomy J. 2001, 93, 1013-1019); (Heaton, E.; Clifton-Brown, J.; Voigt, T.; Jones, M.; Long, S. Mitigation and Adaptation Strategies for Global Change 2004, 9, 433-451; Heaton E.; Voigt, T.; Long, S. P. A quantitative review comparing the yields of two candidate C4 perennial biomass crops in relation to nitrogen, temperature and water. Biomass and Bioenergy, 2004, 27, 21-30; Knauf, M.; Moniruzzaman, M. Lignocellulosic biomass processing: A perspective, Int. Sugar J. 2004, 106, 147-150; Gray, K. A.; Zhao, L.; Emptage, M. Bioethanol, Current Opinion in Chemical Biology 2006, 10, 141-146; Sun, Y.; Cheng, J. Hydrolysis of lignocellulosic materials for ethanol production: a review, Bioresource Technol. 2002, 83, 1-11; and From Niche to Nation: Ethanol Industry Outlook 2006; Renewable Fuels Association Washington D.C.: 2006, p 4.) Numerous agricultural studies throughout Europe have been promising, showing yields ranging from 25 t/acre in Britain and Denmark to 30 t/acre in Spain and Italy. (See e.g., Heaton, E. A., Clifton-Brown, J., Voigt, T. B., Jones, M. B. and Long, S. P. Mitigation and Adaptation Strategies for Global Change 2004, 9: 433-451). Miscanthus also has a higher cellulose content (similar to hardwoods) than most crop residues, thereby increasing the glucose content of the hydrolysis liquid and likely facilitating its bioconversion to ethanol.

There are concerns about Miscanthus as an invasive species. However, one response to these concerns is the lack of seeding in Miscanthus. Miscanthus yields tend to be higher than switchgrass (Panicum vergatum), another energy crop also receiving considerable interest in the United States, although Miscanthus is more expensive to establish than switchgrass. Side by side comparisons of mature stands (established ≧3 years) of these crops have not been reported; however, a quantitative review of the peer reviewed literature has suggested an average Miscanthus yield of 22 Mg ha⁻¹ (97 observations) compared to 10 Mg ha⁻¹ for switchgrass (77 observations). (See e.g., Heaton, E. A., Clifton-Brown, J., Voigt, T. B., Jones, M. B. and Long, S. P. Mitigation and Adaptation Strategies for Global Change 2004, 9: 433-451.) Furthermore, Miscanthus retains high growth efficiency even in cooler climates, thus potentially being preferable in the northern United States (See e.g., Beale, C. V.; Bint, D. A.; Long, S. P. Journal of Experimental Botany 1996, 47, 267 273.) Interest in using biomass derived ethanol has grown strongly in the United States, culminating in a national goal to replace 30% of U.S. gasoline with ethanol by 2030. (See e.g., Energy Policy Act, 2005.) Miscanthus may assist in realizing this goal.

To date, however, little research has been devoted to the pretreatment and enzymatic hydrolysis of Miscanthus. Using a one-step extrusion/NaOH pretreatment process followed by washing the biomass, de Vrije et al. were able to hydrolyze 69% and 38% of the cellulose and hemicellulose respectively into monomeric sugars. (See e.g., de Vrije, T.; de Haas, G.; Tan, G.; Keijsers, E.; Claassen, P. International Journal of Hydrogen Economy 2002, 27, 1381-1390.) This process removes over 75% of the lignin prior to enzymatic hydrolysis, but also removes over 50% of xylan. (See e.g., de Vrije, T.; de Haas, G.; Tan, G.; Keijsers, E.; Claassen, P. International Journal of Hydrogen Economy 2002, 27, 1381-1390.) An alternative approach that does not require removing lignin is the Ammonia Fiber Expansion (AFEX) pretreatment.

### OBJECTS

It is an object of the present invention to provide a process with improved sugar yields from pretreatment of cellosic, i.e., biomass material prior to AFEX process, particularly by the addition of water in suitable and desired quantities and treatment conditions.

These and other objects of the present invention will become increasingly apparent with reference to the following drawings and preferred embodiments.

### SUMMARY OF THE INVENTION

The present disclosure provides for a process by which whole plants are harvested as a biomass and processed together as one unit so that sugars are generated and then optionally fermented to an alcohol which comprises: (a) soaking the biomass in water for a period of time so as to increase the water within the biomass and enhance sugar production from the biomass; (b) treating the plant biomass with concentrated ammonia under pressure in a closed vessel and then relieving the pressure to provide a treated plant biomass with recovery of the ammonia; (c) hydrolyzing the treated plant biomass in the presence of water to sugars using a combination of enzymes which hydrolyze cellulose, hemicellulose and other carbohydrates in the biomass to produce sugars; and (d) optionally fermenting the sugars to produce the alcohol. In an exemplary embodiment, the whole plant comprises both edible grain and non-edible (cell wall) portions of the plant. The whole plant can be selected from the group consisting of corn, wheat and rice. In a further embodiment, the plant cell wall rich portions and grains as the whole plants are harvested at the same time. Further still, the biomass can have a water content in step (d) of between about 0.1 and 2.0 kg water/kg of dry biomass. In an exemplary embodiment, the biomass is charged into the closed vessel reactor with the ammonia in an amount between about 0.2 and 2.0 kg of ammonia/kg of dry biomass. The temperature of the mixture of ammonia and the plant biomass in the closed vessel can be between about 50° C. and 200° C.

The present disclosure provides for a process wherein the biomass in the vessel is at a preselected temperature for a preselected time and then the pressure is released explosively causing disruption of the biomass. In an exemplary embodiment, the pretreated biomass is processed to recover ammonia from the biomass. The sugars in step (c) are separated by filtering the water containing the sugars from the biomass. The sugars are produced from the biomass by cellulase, hemicellulase and amylase during the hydrolysis. Amylase to hydrolyze starch and cellulases and hemicellulases to hydrolyze cell wall components are added in amounts to convert the carbohydrate to fermentable sugars. In a particular embodiment, the alcohol is ethanol. Further still, the disruption of the biomass in step (c) is by an Ammonia Fiber Explosion (also called Ammonia Fiber Expansion) process (AFEX). In a particular embodiment, in step (a) the absorbed ratio of water to biomass is in an amount of about 60 to 233 wt. % on a dry weight basis of the biomass and wherein a water to ammonia ratio is in a range of 0.6:1 to 2.3:3. Further still, the biomass is comminuted before or after the soaking in step (a). Still further, in step (a) the soaking in water is with autoclaving. Further still, a nonionic surfactant is provided with the enzymes in step (c) to increase the hydrolytic activity of the enzymes. Further still, the biomass is washed after the treatment with the ammonia in step (b).

### BRIEF DESCRIPTION OF THE DRAWINGS

FIG. 1 is a schematic diagram illustrating the difference between sprinkling water versus soaking in water prior to an AFEX process treatment.

FIGS. 2A to 2C are photographic illustrations of untreated and pretreated biomass (2A—Miscanthus; 2B—Poplar and 2C—bagasse) before and after water soaking (prior to AFEX): (UT=Untreated, US-AFEX=unsoaked samples, WS-AFEX=water soaked samples).

FIG. 3 is a graph illustrating effect of water soaking and glucan conversion for two batches of poplar after 72 h of enzymatic (60 FPU of cellulase) hydrolysis and at two different AFEX conditions; (US=unsoaked; WS=24-hr water soaked and AC=autoclaving at 120° C. for 60 minutes; prior to AFEX pretreatment respectively).

FIG. 4A shows two different ammonia to biomass (BM) loadings during AFEX process (5 min residence time). The corresponding glucan (white shade) and xylan (gray shade) conversion after 72 (unhatched) and 168-hr (hatched) of enzymatic hydrolysis are shown with duplicate error bars.

FIG. 4B shows glucan and xylan yields after enzymatic hydrolysis for (a) unsoaked (70% on dwb) and (b) 24-hr water soaked Miscanthus (233% moisture on dwb) prior to AFEX (2:1 ammonia to biomass); 5 min residence time at varying temperatures. The corresponding glucan (white shade) and xylan (gray shade) conversion after 72 (unhatched) and 168-hr (hatched) of enzymatic hydrolysis are showing with duplicate error bars.

FIGS. 5A and 5B are graphs showing the effect of varying supplementation of xylanase (5A) and pectinase (5B), along with cellulase and β-glucosidase during enzymatic hydrolysis. Miscanthus was soaked in water for 24-hr prior to AFEX (160° C., 2:1, 233% moisture on dwb, 5 min residence time) and washed prior to hydrolysis. The corresponding glucan (white shade) and xylan (gray shade) conversion after 72 (unhatched) and 168-hr (hatched) of enzymatic hydrolysis are given. T represents Tween-80 supplementation.

FIG. 6 is a graph showing the effect of water soaking and glucan/xylan conversion for bagasse at two different enzyme loading (15 and 60 FPU of cellulase) at a given AFEX condition; (US=unsoaked, WS=water soaked and UT=untreated bagasse prior to AFEX samples respectively).

FIGS. 7A and 7B show Miscanthus, untreated and pretreated samples for biomass conversion; (A) Giant Miscanthus (Miscanthus×giganteus), a hybrid grass that can grow over 12 feet high; (B) Milled Miscanthus samples; untreated (UT), un-soaked and AFEX treated (US-AFEX) and water soaked then AFEX treated (WS-AFEX).

FIGS. 8A and 8B show the effect of varying AFEX conditions on glucan and xylan yields by enzymatic hydrolysis. 8A—the effect of varying temperature at fixed (60%) moisture content (dwb), and 8B—varying moisture content (dwb) for a fixed pretreatment temperature (140° C.), for two different ammonia to biomass (BM) loadings during the AFEX process (5 minutes residence time). The corresponding glucan (white shade) and xylan (grey shade) conversion after 72 (un-hatched) and 168-hr (hatched) of enzymatic hydrolysis are shown with duplicate error bars.

FIGS. 9A and 9B shows glucan and xylan yields after enzymatic hydrolysis for (9A) un-soaked (70% on dwb) and, (9B) 24 hour water soaked Miscanthus (233% moisture on dwb) prior to AFEX (2:1 ammonia to biomass), 5 minutes residence time at varying temperatures. Here, the corresponding glucan (white shade) and xylan (grey shade) conversion after 72 (un17 hatched) and 168-hr (hatched) of enzymatic hydrolysis are shown with duplicate error bars.

FIG. 10 shows glucan and xylan yields after enzymatic hydrolysis for different residence times during the AFEX process (160° C., 2:1 ammonia to biomass and 233% moisture on dwb) for 24-hr water soaked Miscanthus samples. Here, 5, 15 and 30 represents the residence time (in minutes) once the desired temperature is reached during the AFEX process and their corresponding glucan (white shade) and xylan (grey shade) conversions after 72 (un-hatched) and 168-hr (hatched) of enzymatic hydrolysis.

FIGS. 11A and 11B show the effect of varying supplementation of xylanase (11A) and pectinase (11B), along with cellulase and ®-glucosidase during enzymatic hydrolysis. Miscanthus was soaked in water for 24-hr prior to AFEX (160° C., 2:1, 233% moisture on dwb, 5 minutes residence time) and washed prior to hydrolysis. The corresponding glucan (white shade) and xylan (grey shade) conversion after 72 (unhatched) and 168-hr (hatched) of enzymatic hydrolysis are given. Here, T represents Tween-80 supplementation.

FIGS. 12A and 12B show the effect of combinations of xylanase and pectinase, along with cellulase and β-glucosidase during enzymatic hydrolysis for AFEX process (conditions same as in FIG. 5) on Miscanthus sample. The corresponding glucan (white shade) and xylan (grey shade) conversion after 72 (un-hatched) and 168-hr (hatched) of enzymatic hydrolysis are given. Here, (12A) no Tween-80 and (12B) with Tween-80 supplementation respectively.

FIG. 13 is a flow chart showing mass balance during pretreatment and hydrolysis process for Miscanthus. (A) AFEX treatment at 233% moisture content (dwb), 2:1 ammonia loading and 160° C. and (B) untreated sample. Enzymes (C, Cellulase; X, Xylanase; P, Pectinase) and additives (T, Tween 80) used are listed below; 15 FPU/g glucan of cellulase (C), 64 p-NPGU/g glucan of β-glucosidase, 0.56 g/100 g dry Miscanthus of xylanase (X) and Tween-80 (0.35 g/glucan). The sugar yields for AFEX treated Miscanthus were 96% (Glucan) and 81% (Xylan), while for the untreated case they were 5.2% (Glucan) and 3.9% (Xylan).

### DESCRIPTION OF THE PREFERRED EMBODIMENTS

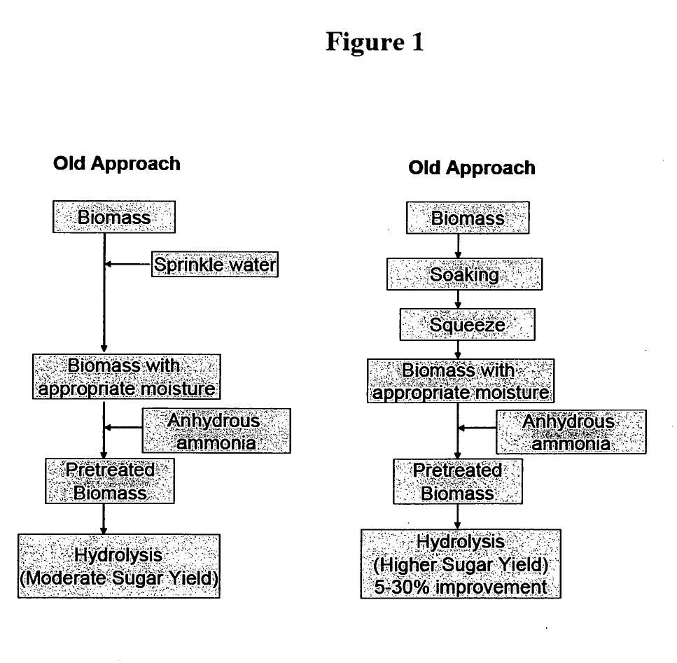

The present disclosure provides for a process by which preferably whole plants are harvested as a biomass and processed together as one unit so that sugars are generated and then preferably fermented to an alcohol. In an exemplary embodiment, the process comprises: soaking the biomass in water for a period of time to enhance sugar production from the biomass. The soaking takes place prior to an ammonia treatment such as AFEX. The biomass material is then hydrolyzed in the presence of water to sugars using a combination of enzymes which hydrolyze cellulose, hemicellulose and other carbohydrates to produce sugars. The sugar can then be fermented to produce the alcohol. In an exemplary embodiment, the whole plant comprises both edible grain and non-edible (cell wall) portions of the plant. It has been found that the water content in three different lignocellulosic biomass materials (poplar, miscanthus and bagasse) improves the accessibility and reactivity of ammonia during the AFEX pretreatment process (FIG. 1). Overall, there was an improvement in the range of 5 to 30% during enzyme hydrolysis for the soaked biomass (233% dwb) followed by AFEX pretreatment compared to biomass with low moisture (60% dwb) followed by AFEX pretreatment (FIG. 1). The present disclosure is shown more clearly by way of the following examples which are provided for illustrative purposes and not intended to limit the scope hereto.

### EXAMPLES 1, 2 and 3

### Materials and Methods

Lignocellulosic Substrate—Miscanthus×giganteus, was provided from Professor Steven P. Long, University of Illinois, Urbana-Champaign and was stored under dry conditions at room temperature until further use. This material was milled using a JT Homoloid mill from the Fitzpatrick Co. with a 3.175 mm diameter sieve. The moisture content was measured using a moisture analyzer (A&D, Model MF-50). Premilled poplar samples were received from NREL, Denver, Colo. and bagasse from MBI, Lansing, Mich.

Compositional Analysis. The NREL standard protocol for acid hydrolysis (LAP-019) was used to determine the glucan and xylan content. The composition of the untreated biomass was as follows: Poplar (45% glucan and 18% xylan), Miscanthus (44% glucan and 19% xylan) and Bagasse (35% glucan and 19% xylan). The conversions reported here were therefore based on these glucan and xylan contents unless samples were soaked or washed.

Soaking. Prior to AFEX treatment, some of the samples were soaked in water for 24 hr to enhance pretreatment effectiveness. Untreated biomass was presoaked in distilled water with a substrate to water loading of 1:10 (w/w). The wash liquid was removed from the substrate by squeezing the slurry through a filtration cloth (Calbiochem, Calif.). The moisture content of the soaked Miscanthus was between 210 and 233% (dry weight basis) after soaking. In a few experiments, the soaked samples were air-dried under a hood to achieve the desired moisture content prior to AFEX pretreatment.

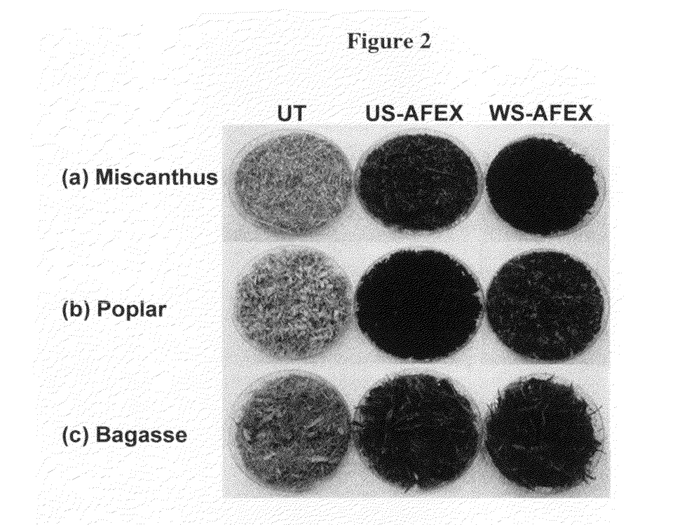

AFEX Treatment. A benchtop reactor was used and consisted of a 300 mL stainless steel pressure vessel (PARR Instrument Co., IL). The vessel was loaded with Miscanthus previously adjusted to the appropriate moisture content. Glass beads were used to help fill the void volume in the vessel. This step reduced the amount of ammonia in the vapor phase during the process. The vessel was clamped shut, and the required amount of ammonia (based on biomass dry weight basis) was injected using a preweighed sample cylinder. A 400 W PARR heating mantle was used to heat the reactor and maintain it at the desired temperature (2° C.) for the necessary residence time. At the completion of the residence time (5-30 min), the pressure was explosively released and the vessel cooled. The biomass was removed from the reactor and left in the hood overnight to evaporate the residual ammonia. FIG. 2 gives details on the visual appearance of three of the untreated and AFEX-treated biomass.

Washing. Some of the AFEX-treated biomass samples were washed in distilled (deionized) water at a (water:substrate) loading of 10:1 (w/w), in a slurry, and mixed for 15 min similar to the protocol reported earlier. (See e.g., Chundawat, S. P. S.; Venkatesh, B.; Dale, B. E. Effect of Particle Size Based Separation of Milled Corn Stover on AFEX Pretreatment and Enzymatic Digestibility. Biotechnol Bioeng. 2007, 96, 219-231.) The wash liquid was removed from the substrate by squeezing the slurry through a filtration cloth (Calbiochem, Calif.).

Enzymatic Hydrolysis. The NREL standard protocol (LAP-009) was followed for enzymatically hydrolyzing the pretreated biomass. All the samples were hydrolyzed in a pH 4.8 citrate buffer at a loading of 1% glucan with the desired cellulase enzyme (Spezyme CP provided by Genencor, CAS 9012-528) at a loading of 15 FPU/(g of glucan) and â-glucosidase (Novo188 by Novozyme) at a loading of 64 p-NPGU/(g of glucan). Certain samples were also hydrolyzed using xylanase (Multifect Xylanase, Genencor), pectinase (Multifect Pectinase, Genencor), and Tween-80 surfactant (0.35 g/g glucan). The samples were hydrolyzed at 50° C., 90 rpm for a period of 168-hr. Sampling was carried out at intervals of 72 and 168-hr to determine glucan and xylan conversions.

HPLC Sugar Analysis. A high-performance liquid chromatography (HPLC) system was used for sugar analysis. The HPLC system consisted of Waters (Milford, Mass.) Pump and Waters 410 refractive index detector and an Aminex HPX-87P carbohydrate analysis column (BioRad, Hercules, Calif.) equipped with a deashing guard cartridge (BioRad). Degassed HPLC grade water was used as the mobile phase at 0.6 mL/min at a column temperature of 85° C. The injection volume was 10/L with a run time of 20 min. Mixed sugar standards were used for quantification of cellobiose and other monosaccharides (glucose, xylose, galactose, arabinose and mannose) in the samples.

### Poplar Hydrolysis.

Among the various sources of biomass, Hybrid poplar (Populus nigra×Populus maximowiczii) is of interest due to its wide availability, high density that reduces cost of transport and improves storage and for its high glucan (˜45%) and xylan (˜18%) content. Two batches of poplar were studied containing high lignin (29%) and lower lignin (21%) levels. Here, we present the results of two different AFEX pretreatment conditions for both the batches of poplar, including some modifications such as soaking in water for 24-hr or autoclaving the poplar sample prior to high and low severity AFEX (100° C., and 180° C.) followed by enzymatic hydrolysis (60 FPU of cellulase loading).

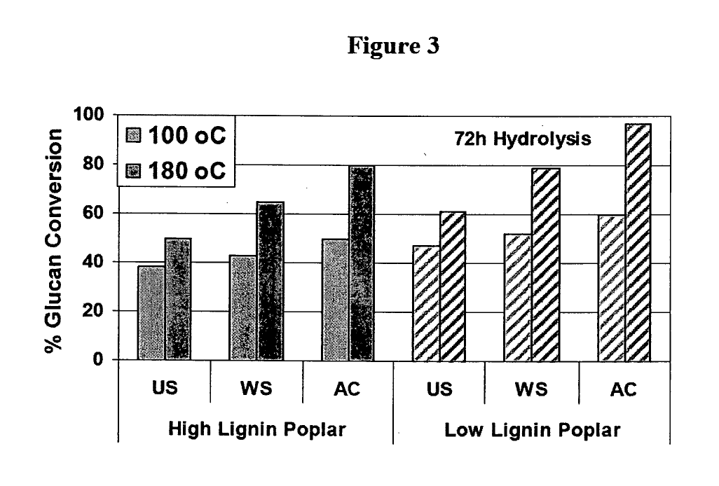

Under these AFEX treatment conditions, conversions (total glucan) ranged from 50-90% (60 FPU) for the different poplar samples at 72-hr, versus 5-10% conversion for untreated samples (FIG. 3), where US is unsoaked, WS is 24 hours soaked and AC is autoclaving at 120° C. for 60 minutes. Looking at the conversion data, we can observe that high lignin poplar was more recalcitrant than the low lignin poplar. Swelling poplar in water prior to AFEX, improves the glucan conversion by 20%. On the other hand, when poplar samples were treating at higher temperature and pressure (120° C. for 60 minutes and 60 psi) (autoclaving in water) prior to AFEX in turn improves the glucan conversion by over 30-35%.

### Miscanthus Hydrolysis

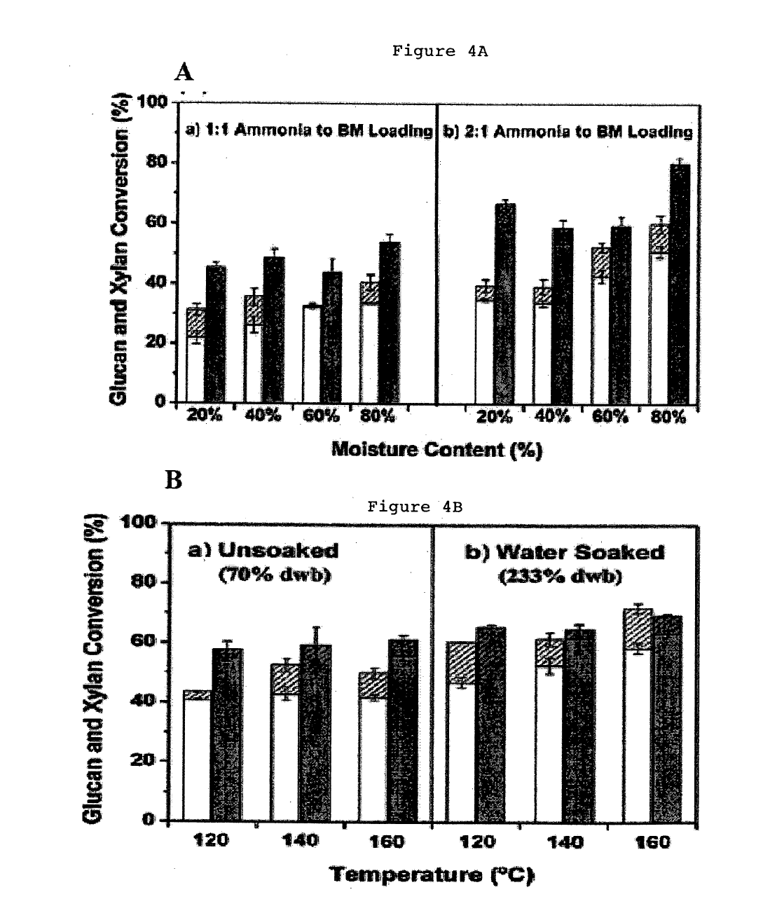

Effect of Moisture Content—FIG. 4A shows the effect of varying the moisture content of the biomass for a constant ammonia loading (either 1:1 or 2:1) and temperature (140° C.). For both 2:1 and 1:1 ammonia loading, increasing the moisture content increased the glucan conversion, with the 2:1 ammonia loading giving higher conversions than the 1:1 loading. The xylan conversion was more variable, peaking at 80% (dwb) moisture and 2:1 (ammonia: biomass) loading. By varying the moisture content for a fixed temperature (140° C.) during the AFEX process, maximum glucan (60%) and xylan (70%) conversions were achieved at 80% moisture (FIG. 4A). These results are comparable to the optimum moisture conditions required for AFEX-treated switchgrass. However, for the 24-hr water soaked sample, the moisture content after removal of the water was 233% (dwb) and was used as is. Up to 10% increase in glucan conversion by soaking Miscanthus in water prior to AFEX (FIG. 4B) was observed.

Effect of Pre-AFEX Soaking and Post-AFEX Washing. Ongoing research indicates the critical role of water in AFEX for some materials. FIG. 4B shows the effect of soaking the sample for 24-hr in water prior to the AFEX treatment. The highest conversion is obtained for 160° C., 2:1 ammonia loading, water-soaked Miscanthus samples (FIG. 4B). The water soaking prevented a drop in glucan conversion at higher temperatures (above 140° C.). Results show that water soaking of Miscanthus prior to AFEX further improves the glucan conversion by 10-15%, as compared to the unsoaked control. Disruption of internal lignocellulosic bonds and by solvent (water) swelling generates internal stresses and improves accessibility to chemicals such as ammonia during the pretreatment process. (See e.g., Ye, D., and Farriol, X., Improving accessibility and reactivity of celluloses of annual plants for the synthesis of methylcellulose, Cellulose, 2005, 12, 507-515; and Boluk, Y. Acid-base interactions and swelling of cellulose fibers in organic liquids. Cellulose 2005, 12, 577-593.) To check whether swelling or the high moisture content (233%, dwb) helped to improve conversion, AFEX pretreatments were done for 24-hr water soaked Miscanthus samples and a dry sample mixed with an equivalent amount of water without soaking. Both the samples showed improved glucan conversion compared to AFEX treatment on Miscanthus with 70% moisture (dwb). These results indicate that the water to ammonia ratio plays an important role during pretreatment. Water helps solvate the ammonia, releasing hydroxyl ions that catalyze the lignin and hemicellulose depolymerization reactions. There appears to be some sort of equilibration time required for the water to gain access to the lignocellulosic ultrastructure (See e.g., Ye, D. 2005 and Boluk, Y. 2005.) All samples were water-soaked for 24-hr prior to AFEX treatment at conditions of 160° C. and 2:1 ammonia loading from this point onward. An AFEX-treated corn stover wash stream was found to contain lignin and sugar degradation compounds that were identified by HPLC-MS/MS and quantified by HPLCUV analyses. It is likely that washing AFEX-treated Miscanthus helps remove some of the degradation products (organic acids, oligosaccharides, and lignophenolics, etc.) such as those shown to be removed in the case of AFEX treated corn stover. (See e.g., Chundawat, S. P. S.; Venkatesh, B.; Dale, B. E. Effect of Particle Size Based Separation of Milled Corn Stover on AFEX Pretreatment and Enzymatic Digestibility. Biotechnol. Bioeng. 2007, 96, 219-231.)

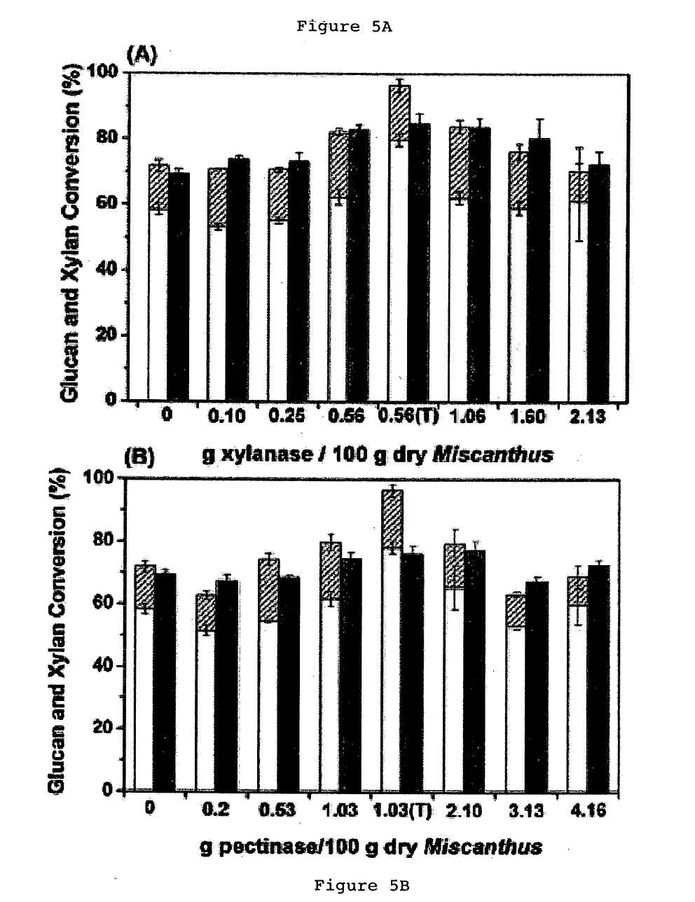

Effect of Additives. FIGS. 5A and 5B show the effect of additives (varying concentrations of xylanases and/or pectinases) on enzymatic hydrolysis using AFEX-treated (160° C., 2:1 ammonia loading, presoaked) Miscanthus. Xylanase and pectinase are known to hydrolyze xylan and pectin hemicellulose oligomers present surrounding the cellulosic microfibrils. (See e.g., Teymouri, F., Laureano-Perez, L., and Alizadeh, H., and Dale, B. E., Optimization of the ammonia fiber explosion (AFEX) treatment parameters for enzymatic hydrolysis of corn stover, Bioresource Technol., 2005, 96, 20142018; and Foster, B. L.; Dale, B. E.; Doran-Peterson, J. B. Enzymatic hydrolysis of ammonia treated sugar beet pulp. Appl. Biochem. Biotechnol. 2001, 91-93, 269-82.) These synergistic actions of cellulase, xylanase, and pectinase along with β-glucosidase result in higher sugar yields. However, the optimum synergistic enzyme concentrations taper off at higher loadings, probably due to nonspecific binding of enzymes to substrate. (See e.g., Jeoh, T.; Wilson, D. B.; Walker, L. P. Cooperative and competitive binding in synergistic mixtures of Thermobifida fusca cellulases Cel5A, Cel6B, and Cel9A. Biotechnol. Prog. 2002, 18, 760-769.) It is believed C4 warm-season grasses are very low in pectin. The crude pectinase preparation which was used in these experiments is expected to contain multiple other activities. Tween-80 helps to further improve the glucan and xylan yields. Tween-80 is a nonionic surfactant that prevents irreversible binding of lignin to hydrolytic enzymes. (See e.g., Kim, S. B.; Kim, H. J.; Kim, C. J. Enhancement of the enzymatic digestibility of waste newspaper using Tween. Appl. Biochem. Biotchnol. 2006, 129-132, 486-495.) For 24-hr water soaked followed by AFEX-pretreated Miscanthus, with the combination of cellulase, β-glucosidase, pectinase, or xylanase along with Tween-80, a glucan and xylan conversion of 90-95 and 80-85%, respectively, was achieved. However, when both xylanase and pectinase were added together with cellulase, they did not show a greater impact on the conversions (FIGS. 5(A) and 5(B)). Glucose conversions were actually slightly lower (85-88%) than those achieved when adding xylanase or pectinase alone. This could be due to competition between pectinase and xylanase for a similar substrate or due to nonspecific binding of enzyme on the substrate (See e.g., Jeoh, T.; Wilson, D. B.; Walker, L. P. Cooperative and competitive binding in synergistic mixtures of Thermobifida fusca cellulases Cel5A, Cel6B, and Cel9A. Biotechnol. Prog. 2002, 18, 760-769.)

Bagasse Hydrolysis

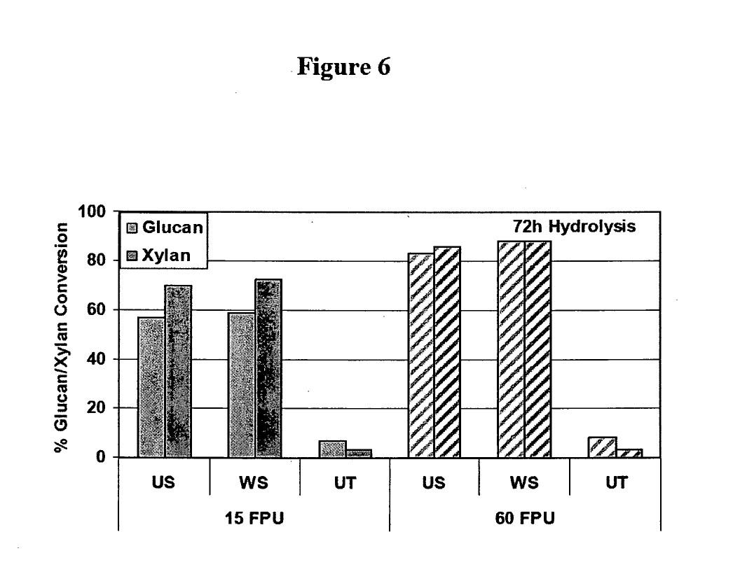

Sugarcane bagasse represents one of the major lignocellulosic materials as a biofuel feedstock considered in most tropical countries since it has high carbohydrate content. It is readily available at the sugar mill plant sites, and the cost for harvest, transport and storage has been borne by the sugar production. (See e.g., Pandey, A and Soccol, C. R., Economic utilization of crop residues for value addition: a futuristic approach. J. Sci. Ind. Res. 2000, 59, 12-22.) The use of bagasse for ethanol production would contribute to the diversification of the sugar industry, which is an urgent requirement for the sugarcane-based economies. (See e.g., Ga'lvez, L. O., Diversified production of the sugarcane agro-industry. In: Ga'lvez, L. O. (Ed.), Handbook of Sugarcane Derivatives, third ed. ICIDCA, Havana, (2000) pp. 3-17.) There is a 5 to 7 fold increase in glucan conversion by pretreating the bagasse by AFEX. For unsoaked sample, highest conversion is obtained at 100° C., 2:1 ammonia loading. Similar conditions were used for soaked bagasse. FIG. 6 shows the effect of soaking bagasse in water, where US is unsoaked, WS is water soaked and UT is untreated.

### Example 4

The effect of pretreatment and enzymatic saccharification of Miscanthus to produce fermentable sugars was further investigated. Sugar yields during enzymatic hydrolysis from ammonia fiber expansion (AFEX) pretreated Miscanthus is investigated. Pretreatment conditions including temperature, moisture, ammonia loading, residence time and enzyme loadings are varied to maximize hydrolysis yields. In addition, further treatments such as soaking the biomass prior to AFEX as well as washing the pretreated material were also attempted to improve sugar yields. The optimal AFEX conditions determined were 160° C., 2:1 (w/w) ammonia to biomass loading, 233% moisture (dry weight basis), and 5 minute reaction time for water soaked Miscanthus. Approximately 96% glucan and 81% xylan conversions were achieved after 168-hr enzymatic hydrolysis at 1% glucan loading using 15 FPU/g glucan of cellulase and 64 p-NPGU/g glucan of beta-glucosidase along with xylanase and tween-80 supplementation. A mass balance for the AFEX pretreatment and enzymatic hydrolysis process is presented.

In an exemplary embodiment, this process contacts biomass with concentrated ammonia at temperatures of 70-180° C. and pressure ranges between 200 to 1000 psi. After a brief residence time, the pressure is explosively released, effectively disrupting the structure of the biomass. AFEX decrystallizes cellulose, partially hydrolyzes hemicellulose, and depolymerizes lignin. (See e.g., Mosier, N.; Wyman, C.; Dale, B.; Elander, R.; Lee, Y.; Holtzapple, M.; Ladisch, M. Bioresource Technology 2005, 96, 673-686.)

Previous work has shown that AFEX followed by enzymatic hydrolysis gives near theoretical yields of glucose on different types of agricultural residue. (See e.g., Sulbaran-de-Ferrer, B.; Aristguieta, M.; Dale, B.; Ferrer, A; Ojeda-de-Rodriguez, G. Enzymatic hydrolysis of ammonia-treated rice straw. Appl. Biochem. Biotechnol. 2003, 105-108, 155-164; and Teymouri, F.; Laureano-Perez, L.; Alizadeh, H.; Dale, B. E. Optimization of the ammonia fiber explosion (AFEX) treatment parameters for enzymatic hydrolysis of corn stover. Bioresource Technol. 2005, 96, 2014-2018.) Previous work with other herbaceous energy crops, namely switchgrass (Alizadeh, H.; Teymouri, F.; Gilbert, T.; Dale, B. Applied Biochemistry and Biotechnology 2005, 121-124, 1133-1141) and dwarf elephant grass (Ferrer, A.; Byers, F. M.; Sulbarán De Ferrer, B.; Dale, B. E.; Aiello, C. Applied Biochemistry and Biotechnology, 2000, 8486, 163-179), has shown high yields of both glucose and xylose after AFEX pretreatment.

Maximizing sugar yields during enzymatic hydrolysis for AFEX pretreated Miscanthus is desired. Pretreatment process conditions such as temperature, moisture, ammonia loading and residence time and enzyme loadings are varied to determine the effect on enzymatic hydrolysis. In addition, further treatments such as water soaking the biomass prior to AFEX as well as washing the material afterwards are also attempted to improve sugar yields. A mass balance for the AFEX pretreatment and enzymatic hydrolysis process is presented.

### Materials and Methods

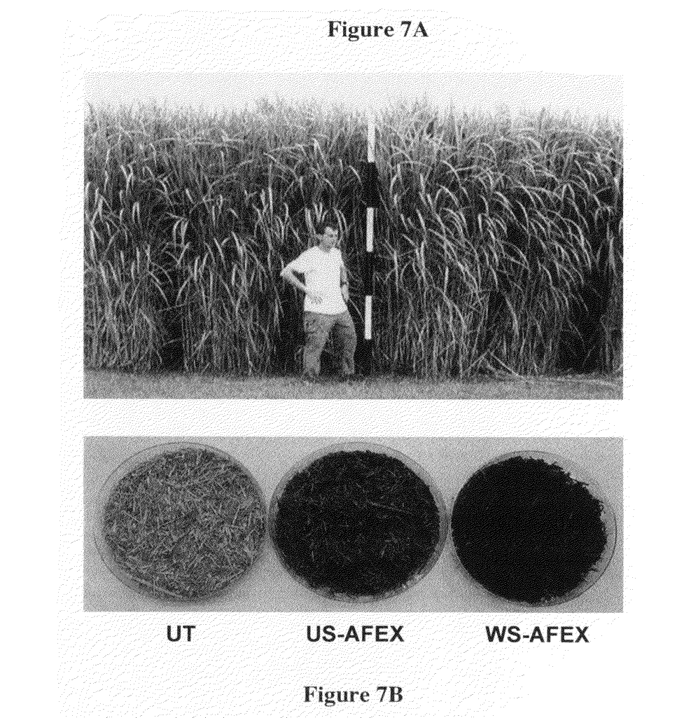

Lignocellulosic substrate. As previously discussed (Example 3), Miscanthus×giganteus, was harvested and provided from Professor Steven P. Long, University of Illinois, Urbana-Champaign (FIG. 7A) and was stored under dry conditions at room temperature until further use. This material was milled using a JT Homoloid mill from the Fitzpatrick Company with a 3.175 mm diameter sieve. The moisture content was measured using a moisture analyzer (A&D, Model MF-50).

Compositional Analysis. The NREL standard protocol for Acid Hydrolysis (LAP-019) was used to determine the glucan and xylan content. The composition of the untreated Miscanthus was found to be 44% glucan and 19% xylan. The conversions reported here were therefore based on these glucan and xylan contents unless samples were soaked or washed. The washing step after AFEX pretreatment results in increasing the glucan and xylan content to 49% and 21% respectively because of removal of soluble lignin components and water soluble extractives.

Soaking. Prior to the AFEX treatment, some of the samples were soaked in water for 24-hr to enhance pretreatment effectiveness. Untreated biomass was presoaked in distilled water with a substrate to water loading of 1:10 (w/w). The wash liquid was removed from the substrate by squeezing the slurry through a filtration cloth (Calbiochem, Calif.). The moisture content of the soaked Miscanthus was between 210-233% (dry weight basis) after soaking. In a few experiments, the soaked samples were air dried under a hood to achieve the desired moisture content prior to AFEX pretreatment.

AFEX Treatment. A bench-top reactor consisted of a 300 ml stainless steel pressure vessel (PARR Instrument Co, Ill.). The vessel was loaded with Miscanthus previously adjusted to the appropriate moisture content. Glass beads were used to help fill the void volume in the vessel. This step reduced the amount of ammonia in the vapor phase during the process. The vessel was clamped shut and the required amount of ammonia (based on biomass dry weight basis) was injected using a pre-weighed sample cylinder. A 400 W PARR heating mantle was used to heat the reactor and maintain it at the desired temperature (+/−2° C.) for the necessary residence time. At the completion of the residence time (5-30 min.), the pressure was explosively released and the vessel cooled. The biomass was removed from the reactor and left in the hood overnight to evaporate the residual ammonia. FIG. 1B gives details on the visual appearance of untreated and AFEX treated Miscanthus.

Washing. Some of the AFEX treated biomass samples were washed in distilled (deionized) water at a (water: substrate) loading of 10:1 (w/w), in a slurry and mixed for 15 minutes similar to the protocol reported earlier. (See e.g., Chundawat S. P. S., Venkatesh, B.; Dale, B. E. Effect of Particle Size Based Separation of Milled Corn Stover on AFEX Pretreatment and Enzymatic Digestibility. Biotech Bioeng. 2007, 96, 219-231.) The wash liquid was removed from the substrate by squeezing the slurry through a filtration cloth (Calbiochem, Calif.).

Enzymatic Hydrolysis. The NREL standard protocol (LAP-009) was followed for enzymatically hydrolyzing the pretreated biomass. All the samples were hydrolyzed in a pH 4.8 citrate buffer at a loading of 1% glucan with the desired cellulase enzyme (Spezyme CP provided by Genencor, CAS 9012-528) at a loading of 15 FPU/g of glucan and β-glucosidase (Novo188 by Novozyme) at a loading of 64 p-NPGU/g of glucan. Certain samples were also hydrolyzed using xylanase (Multifect Xylanase, Genencor), pectinase (Multifect Pectinase, Genencor) and Tween-80 surfactant (0.35 g/g glucan). The samples were hydrolyzed at 50° C., 90 rpm for a period of 168-hr. Sampling was carried out at intervals of 72 and 168-hr to determine glucan and xylan conversions.

HPLC Sugar Analysis: A high performance liquid chromatography (HPLC) system was used for sugar analysis. The HPLC system consisted of Waters (Milford, Mass.) Pump and Waters 410 refractive index detector, an Aminex HPX-87P carbohydrate analysis column (BioRad, Hercules, Calif.) equipped with a deashing guard cartridge (BioRad). Degassed HPLC grade water was used as the mobile phase at 0.6 ml/min at a column temperature of 85° C. The injection volume was 10 μl with a run time of 20 min. Mixed sugar standards were used for quantification of cellobiose and other monosaccharides (glucose, xylose, galactose, arabinose and mannose) in the samples.

### Results and Discussion

Both untreated and AFEX treated Miscanthus are shown in FIG. 7B. The untreated Miscanthus was hydrolyzed and gave less than 5-10% glucan and xylan conversion. AFEX pretreatment significantly increased the enzymatic hydrolysis of Miscanthus, giving between 30-90% conversions, depending on the pretreatment parameters. Therefore, a detailed study of the effect of various pretreatment parameters such as moisture, ammonia loading and temperature on the hydrolysis yields was undertaken.

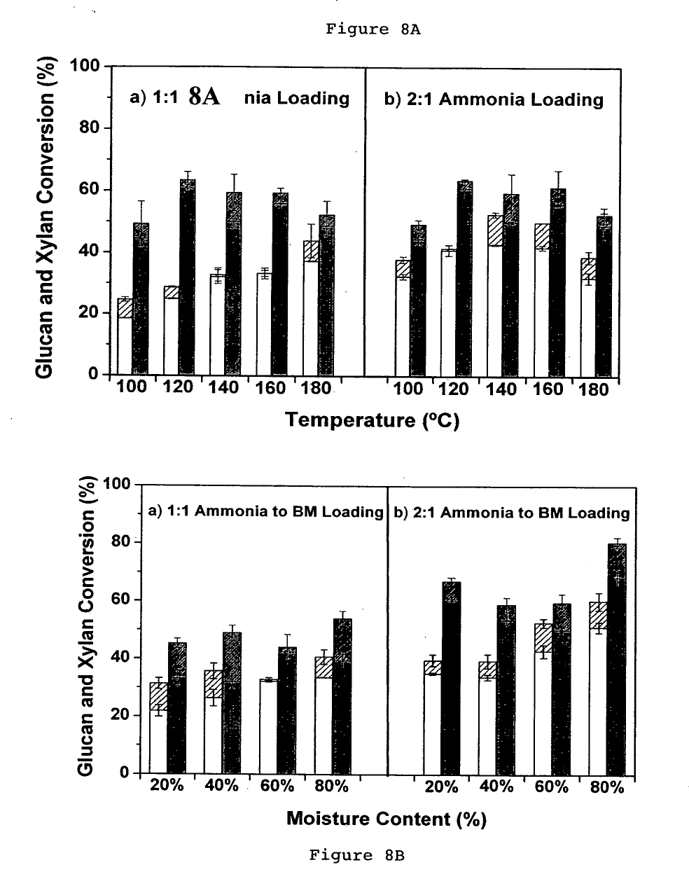

Effect of Temperature and Ammonia Loading. FIG. 8A shows the effect of increasing pretreatment temperature (100° C.-180° C.) on the enzymatic hydrolysis yields of AFEX-treated Miscanthus, while holding the ammonia to biomass ratio constant either at 1:1 or 2:1 and 60% moisture content (dwb-dry weight basis). The higher ammonia level is clearly beneficial in increasing the maximum glucan conversion at all temperatures. However, the increase in glucan conversion levels off at temperatures above 140° C. for 2:1 ammonia loading, unlike the 1:1 ammonia loading. At higher temperatures (180° C.) for the 2:1 ammonia loading the glucan conversion drops off substantially. This may be a result of charring or alkali enhanced degradation of the biomass at high temperatures. The xylan conversion, on the other hand, is fairly constant across 120° C.-160° C. in the 2:1 case and only drops off at 180° C. This result indicates that the limiting factor for higher glucan-xylan conversions might also be the optimal combination of enzymes along with pretreatment severity. A 2:1 ammonia loading was selected for all subsequent experiments (FIG. 8A). It is likely that ammonia loadings can be reduced, but these experiments have not yet been done.

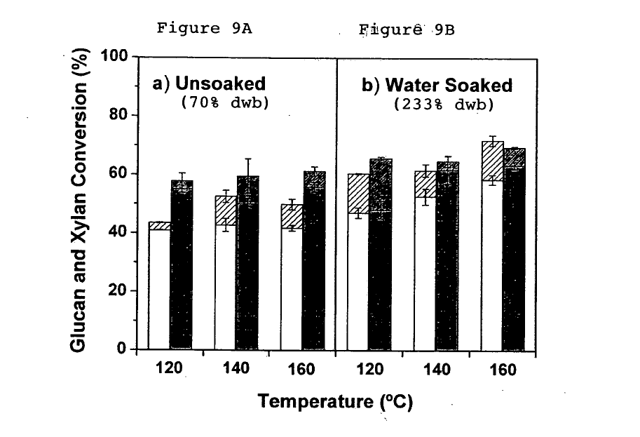

Effect of Moisture Content FIG. 8B shows the effect of varying the moisture content of the biomass for a constant ammonia loading (either 1:1 or 2:1) and temperature (140° C.). For both 2:1 and 1:1 ammonia loading, increasing the moisture content increased the glucan conversion, with the 2:1 ammonia loading giving higher conversions than 1:1 loading. The xylan conversion was more variable, peaking at 80% (dwb) moisture and 2:1 (ammonia: biomass) loading. By varying the moisture content for a fixed temperature (140° C.) during the AFEX process, maximum glucan (60%) and xylan (70%) conversion were achieved at 80% moisture (FIG. 8B). These results are comparable to the optimum moisture conditions required for AFEX treated switchgrass. (See e.g., Alizadeh, H.; Teymouri, F.; Gilbert, T.; Dale, B. Applied Biochemistry and Biotechnology 2005, 121-124, 1133-1141.) However, for the 24-hr water soaked sample, the moisture content after removal of the water was 233% (dwb) and was used as is. Up to 10% increase in glucan conversion was observed by soaking Miscanthus in water prior to AFEX (FIG. 9).

Effect of pre-AFEX Soaking and post-AFEX Washing. Ongoing research indicates the critical role of water in AFEX for some materials. FIG. 9 shows the effect of soaking the sample for 24-hr in water prior to the AFEX treatment. The highest conversion is obtained for 160° C., 2:1 ammonia loading, water soaked Miscanthus samples. The water soaking prevented a drop in glucan conversion at higher temperatures (above 140° C.). Results show that water soaking of Miscanthus prior to AFEX further improves the glucan conversion by 10-15%, as compared to the unsoaked control. Disruption of internal lignocellulosic bonds and by solvent (water) swelling will generate internal stresses and might improve accessibility to chemicals like ammonia during the pretreatment process. (See e.g., Ye, D.; Farriol, X. Improving accessibility and reactivity of celluloses of annual plants for the synthesis of methylcellulose, Cellulose, 2005, 12, 507-515; and Boluk, Y. Acid-base interactions and swelling of cellulose fibers in organic liquids, Cellulose, 2005, 12, 577-593.) To check whether swelling or the high moisture content (233%, dwb) helped to improved conversion, AFEX pretreatments were done for 24-hr water soaked Miscanthus samples and a dry sample mixed with an equivalent amount of water without soaking. Both the samples showed improved glucan conversion compared to AFEX treatment on Miscanthus with 70% moisture (dwb). These results indicate that the water to ammonia ratio plays a very important role during pretreatment. Water will help solvate the ammonia, releasing hydroxyl ions that catalyze the lignin and hemicellulose depolymerization reactions. There appears to be some sort of equilibration time required for the water to gain access to the lignocellulosic ultrastructure. (See e.g., Ye, D.; 2005; and Boluk, Y. 2005.) All samples were water soaked for 24-hr prior to AFEX treatment at conditions of 160° C. and 2:1 ammonia loading from this point onwards.

An AFEX-treated corn stover wash stream was found to contain lignin and sugar degradation compounds that were identified by HPLC-MS/MS and quantified by HPLCUV analyses. Therefore it is likely that washing AFEX treated Miscanthus helps remove some of the degradation products (organic acids, oligosaccharides, ligno-phenolics, etc) such as those shown to be removed in the case of AFEX-treated corn stover. (See e.g., Chundawat S. P. S., Venkatesh, B.; Dale, B. E. Effect of Particle Size Based Separation of Milled Corn Stover on AFEX Pretreatment and Enzymatic Digestibility. Biotech Bioeng. 2007, 96, 219-231).

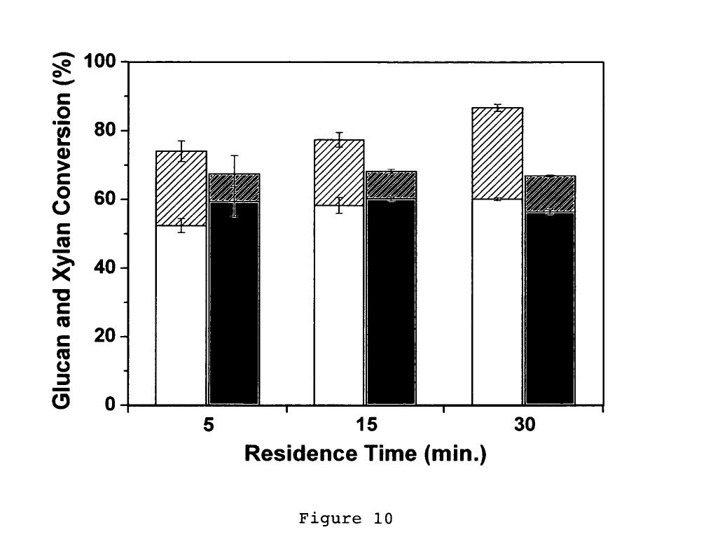

Effect of Residence Time. FIG. 10 indicates the effect of residence time on the AFEX process. For the optimization of the moisture content, ammonia loading and temperature, as shown previously, the residence time was kept fixed at 5 minutes. However, a longer residence time might help improve the overall conversions considering the recalcitrant nature of this material. By increasing the AFEX residence time from 5 to 30 minutes, for a 160° C., 2:1 ammonia loading presoaked Miscanthus sample, the glucan conversion increased significantly. However, there was no significant improvement in the xylan yield. Increasing the residence time to 30 minutes seemed to have the most beneficial effect beyond which little or no improvement was observed.

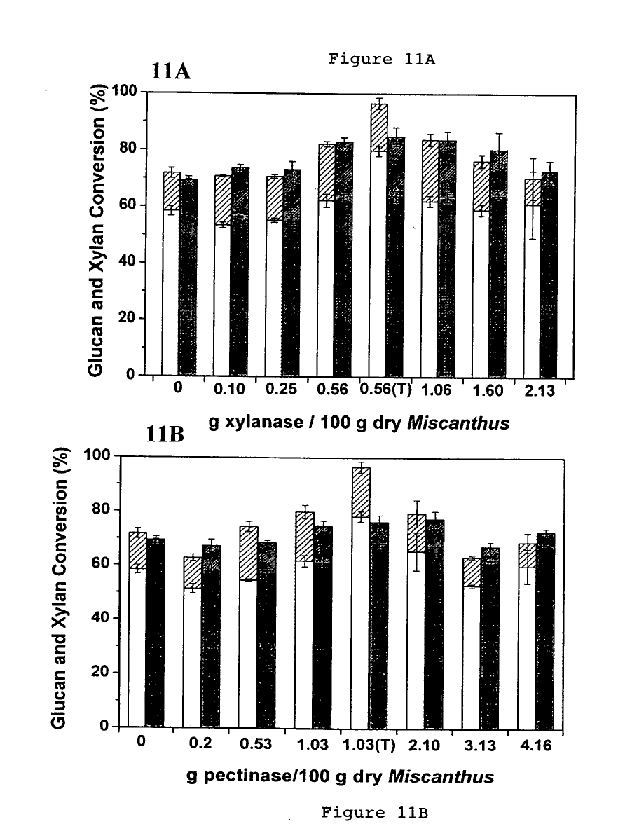

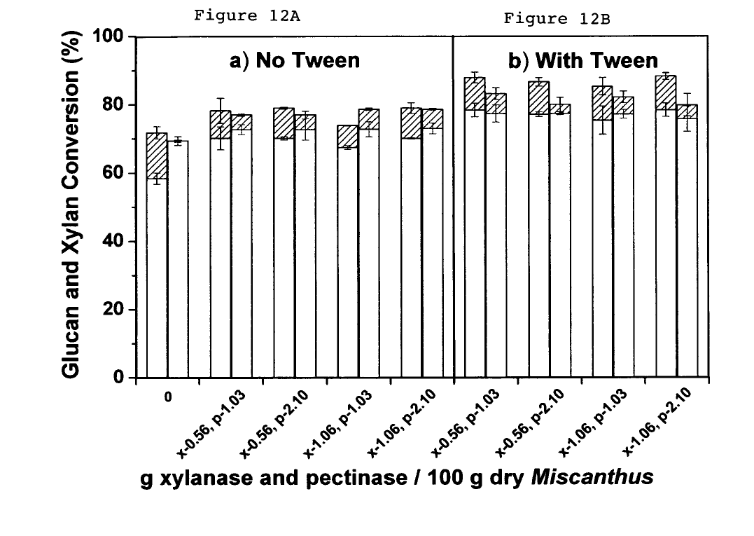

Effect of Additives. FIGS. 11A, 11B, 12A and 12B show the effect of additives (varying concentrations of xylanases and/or pectinases) on enzymatic hydrolysis using AFEX treated (160° C., 2:1 ammonia loading, presoaked) Miscanthus. Xylanase and pectinase are known to hydrolyze xylan and pectin hemicellulose oligomers present surrounding the cellulosic microfibrils. (See e.g., Teymouri, F.; Laureano-Perez, L.; Alizadeh, H.; Dale, B. E. Optimization of the ammonia fiber explosion (AFEX) treatment parameters for enzymatic hydrolysis of corn stover. Bioresource Technol. 2005, 96, 2014-2018; and Foster, B. L.; Dale, B. E.; Doran-Peterson, J. B. Enzymatic hydrolysis of ammonia treated sugar beet pulp. Appl Biochem Biotechnol. 2001, 91-93, 269-82.) These synergistic actions of cellulase, xylanase, and pectinase along with beta-glucosidase result in higher sugar yields. However, the optimum synergistic enzyme concentrations taper off at higher loadings, probably due to non-specific binding of enzymes to substrate. (See e.g., Jeoh, T.; Wilson, D. B.; Walker, L. P. Cooperative and competitive binding in synergistic mixtures of Thermobifida fusca cellulases Cel5A, Cel6B, and Cel9A. Biotechnol Prog. 2002, 18, 760-769.) It is believed C4 warm season grasses are very low in pectin. The crude pectinase preparation which was used in these experiments is expected to contain multiple other activities. Tween-80 helps to further improve the glucan and xylan yields. Tween-80 is a nonionic surfactant that prevents irreversible binding of lignin to hydrolytic enzymes. (See e.g., Kim, S. B.; Kim, H. J.; Kim, C. J. Enhancement of the enzymatic digestibility of waste newspaper using Tween. Appl. Biochem. Biotchnol. 2006, 129-132, 486-495.)

For 24-hr water soaked followed by AFEX pretreated Miscanthus, with the combination of cellulase, beta-glucosidase, pectinase or xylanase along with Tween-80, a glucan and xylan conversion of 90-95% and 80-85%, respectively, was achieved. However when both xylanase and pectinase were added together with cellulase they did not show a greater impact on the conversions (FIGS. 12A and 12B). Glucose conversions were actually slightly lower (85-88%) than those achieved when adding xylanase or pectinase alone. This could be due to competition between pectinase and xylanase for a similar substrate or due to non specific binding of enzyme on the substrate. (See e.g., Jeoh, T.; Wilson, D. B.; Walker, L. P. Cooperative and competitive binding in synergistic mixtures of Thermobifida fusca cellulases Cel5A, Cel6B, and Cel9A. Biotechnol Prog. 2002, 18, 760-769.)

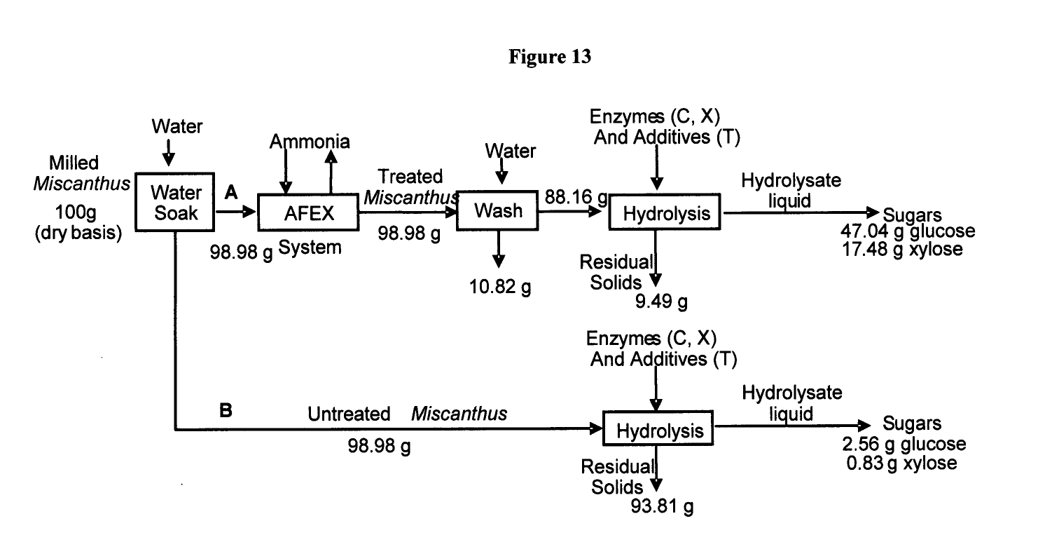

Mass Balance. In order to better understand the process, a detailed mass balance was done for each step (FIG. 13). The samples were soaked prior to AFEX treatment and the optimal AFEX conditions used were 160° C., 2:1 ammonia loading, and 5 min. reaction time. Following AFEX, the washing step resulted in a 10-11% mass loss of the original untreated material. In comparison, the soaking process prior to AFEX treatment resulted in a 1% mass loss. The AFEX process itself was assumed not to have added material to or removed material from the biomass, based on our previous work. (See e.g., Teymouri, F.; Laureano-Perez, L.; Alizadeh, H.; Dale, B. E. Optimization of the ammonia fiber explosion (AFEX) treatment parameters for enzymatic hydrolysis of corn stover. Bioresource Technol. 2005, 96, 2014-2018.) An enzyme loading of 15 FPU/g glucan of cellulase (Spezyme CP), 64 p-NPGU/g glucan of β-glucosidase (Novozyme 188) with xylanase (0.53 g/100 g of Miscanthus) and tween 80 (0.35 g/g glucan) were used to hydrolyze the AFEX treated and untreated biomass. The samples were hydrolyzed at 50° C., 200 rpm for a period of 168-hr. Approximately 96% glucan and 81% xylan conversion was achieved at the end of 168-hr for 1% glucan loading based enzymatic hydrolysis. A 20 fold increase in the glucose and xylose concentration is seen for the AFEX treated hydrolysate compared to the untreated hydrolysate (FIG. 13). The overall glucan conversions combinations of xylanase and pectinase added together are lower (85-88%) than conversions obtained using these same enzymes added sequentially (FIGS. 11A, 11B, 12A and 12B).

### CONCLUSION

AFEX pretreatment parameters were varied to screen for appropriate AFEX conditions for Miscanthus. Two different ammonia to biomass loadings (i.e., 1:1 and 2:1, ammonia to biomass (w/w)) were used, along with varying temperatures (100-180° C.) and moisture contents (20-80% dwb). Water soaking the biomass prior to AFEX (pre-AFEX soaking) followed by washing the treated material (post-AFEX washing) and hydrolyzing with cellulolytic enzymes supplemented by additives (i.e. xylanase and/or pectinase and tween-80) results in nearly theoretical glucan and xylan conversions. Glucan and xylan conversions close to 90-95% and 80-85%, respectively, were possible upon hydrolysis of AFEX treated Miscanthus using cellulases (15 FPU/g of glucan) and other additives, such as xylanase (0.53 g/100 g of Miscanthus) or pectinase (1.03 g/100 g of Miscanthus) and Tween-80 (0.35 g/g of glucan). Additional improvements in the AFEX process and better enzyme mixtures should help improve the glucan and xylan conversions at lower pretreatment severities.

This research was conducted at the Biomass Conversion Research Lab (BCRL), Michigan State University, supported by funds from Michigan State University Research Foundation. Special thanks to Dr. Steven P. Long for authorizing use of the Miscanthus photograph and for providing the Miscanthus material and to MBI, International (Lansing, Mich.) for grinding the biomass.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

We claim:

1. A process by which whole plants are harvested as a biomass and processed together as one unit so that sugars are generated and then optionally fermented to an alcohol which comprises:
(a) soaking the biomass in water for a period of time so as to increase the water within the biomass and to enhance sugar production from the biomass;
(b) treating the plant biomass with concentrated ammonia under pressure in a closed vessel and then relieving the pressure to provide a treated plant biomass with recovery of the ammonia;
(c) hydrolyzing the treated plant biomass in the presence of water to sugars using a combination of enzymes which hydrolyze cellulose, hemicellulose and other carbohydrates in the biomass to produce sugars; and
(d) optionally fermenting the sugars to produce the alcohol.

2. The process of claim 1 wherein the whole plant comprises both edible grain and non-edible (cell wall) portions of the plant.

3. The process of claim 2 wherein the whole plant is selected from the group consisting of corn, wheat and rice.

4. The process of claim 1 wherein plant cell wall rich portions and grains as the whole plants are harvested at the same time.

5. The process of any one of claims 1, 2, 3, or 4 wherein the biomass has a water content in step (d) of between about 0.1 and 2.0 kg water/kg of dry biomass.

6. The process of any one of claims 1, 2, 3, or 4 wherein the biomass is charged into the closed vessel reactor with the ammonia in an amount between about 0.2 and 2.0 kg of ammonia/kg of dry biomass.

7. The process of claim 1 wherein the temperature of the mixture of ammonia and the plant biomass in the closed vessel is between about 50° C. and 200° C.

8. The process of claim 1 wherein the biomass in the vessel is at a preselected temperature for a preselected time and then the pressure is released explosively causing disruption of the biomass.

9. The process of claim 8 wherein the pretreated biomass is processed to recover ammonia from the biomass.

10. The process of claim 1 wherein the sugars in step (c) are separated by filtering the water containing the sugars from the biomass.

11. The process of claim 1 wherein the sugars are produced from the biomass by cellulase, hemicellulase and amylase during the hydrolysis.

12. The process of claim 1 wherein amylase to hydrolyze starch and cellulases and hemicellulases to hydrolyze cell wall components are added in amounts to convert the carbohydrate to fermentable sugars.

13. The process of claim 1 wherein the alcohol is ethanol.

14. The process of claim 1 wherein the disruption of the biomass in step (c) is by an Ammonia Fiber Explosion (also called Ammonia Fiber Expansion) process (AFEX).

15. The process of claim 1 wherein in step (a) the absorbed ratio of water to biomass is in an amount of about 60 to 233 wt. % on a dry weight basis of the biomass and wherein a water to ammonia ratio is in a range of 0.6:1 to 2.3:3.

16. The process of claim 1 wherein the biomass is comminuted before or after the soaking in step (a).

17. The process of any one of claims 1, 2, 3, or 4 wherein in step (a) the soaking in water is with autoclaving.

18. The process of any one of claims 1, 2, 3, or 4 wherein a nonionic surfactant is provided with the enzymes in step (c) to increase the hydrolytic activity of the enzymes.

19. The process of any one of claims 1, 2, 3 or 4 wherein the biomass is washed after the treatment with the ammonia in step (b).
